(19) United States
(12) Patent Application Publication
(10) Pub. No.: US 2011/0319710 A1
(43) Pub. Date: Dec. 29, 2011

(54) SCOPE PROTECTION FOR ENDOSCOPIC DEVICES

(76) Inventors: Molly Phillips-Hungerford, Somerville, MA (US); Benjamin E. Morris, Jeffersonville, IN (US); John Lane, Manchester, NH (US)

(21) Appl. No.: 13/166,979

(22) Filed: Jun. 23, 2011

Related U.S. Application Data
(60) Provisional application No. 61/359,117, filed on Jun. 28, 2010.

(51) Int. Cl. A61B 1/018

(52) U.S. Cl. 600/106; 600/123

(57) ABSTRACT

A medical device for insertion through a channel of an endoscope comprises a sheath forming a smooth outer surface over a first abrasive portion of a length of the device. A second portion of the length of the device remains uncovered by the sheath.

### PRIORITY CLAIM

The present invention claims the priority to the U.S. Provisional Application Ser. No. 61/359,117 entitled “Scope Protection for Endoscopic Devices” filed on Jun. 28, 2010. The specification of the above-identified application is incorporated herewith by reference.

### BACKGROUND

Most endoscopic devices are often covered via, for example, an outer sheath, a plastic jacket or a heat shrunk layer, to protect the scope from damage as the devices are slid therethrough. Current endoscopic devices generally include coverings extending over the entire lengths of the portions of the devices which will be inserted into the endoscope. These lengths often range between approximately 155 cm and 235 cm. However, such coverings can limit the flexibility and functionality of the devices, especially when the devices must navigate through tortuous paths such as, for example, when used in a duodenoscope.

### SUMMARY OF THE INVENTION

The present invention is directed to a medical device for insertion through a channel of an endoscope comprising a sheath forming a smooth outer surface over a first abrasive portion of a length of the device, a second portion of the length of the device remaining uncovered by the sheath.

### BRIEF DESCRIPTION OF THE DRAWINGS

FIG. 1 shows a side view of a system according to a first exemplary embodiment of the present invention;

FIG. 2 shows a side view of the system of FIG. 1, in a deployed configuration;

FIG. 3 shows a side view of a system according to a second exemplary embodiment of the present invention;

FIG. 4 shows a side view of a system according to a third exemplary embodiment of the present invention.

### DETAILED DESCRIPTION

The present invention may be further understood with reference to the following description and the appended drawings, wherein like elements are referred to with the same reference numerals. The present invention relates to endoscopic devices and, in particular, relates to a covering for endoscopic devices. Exemplary embodiments of the present invention provide a mini sheath extending over only abrasive portions of an endoscopic device to minimize damage to the endoscope while maintaining the flexibility and functionality of the device. Although the exemplary embodiments specifically describe a hemostatic clipping device, it will be understood by those of skill in the art that the present invention may be used for any endoscopic devices such as, for example, tissue stapling devices and resectioning devices. It will also be understood by those of skill in the art that the present invention may be used for any device that is inserted through an introducer, a sheath or other similar element such as, for example, biopsy forceps, graspers, hemostatic clips, tissue clamps, baskets, etc.

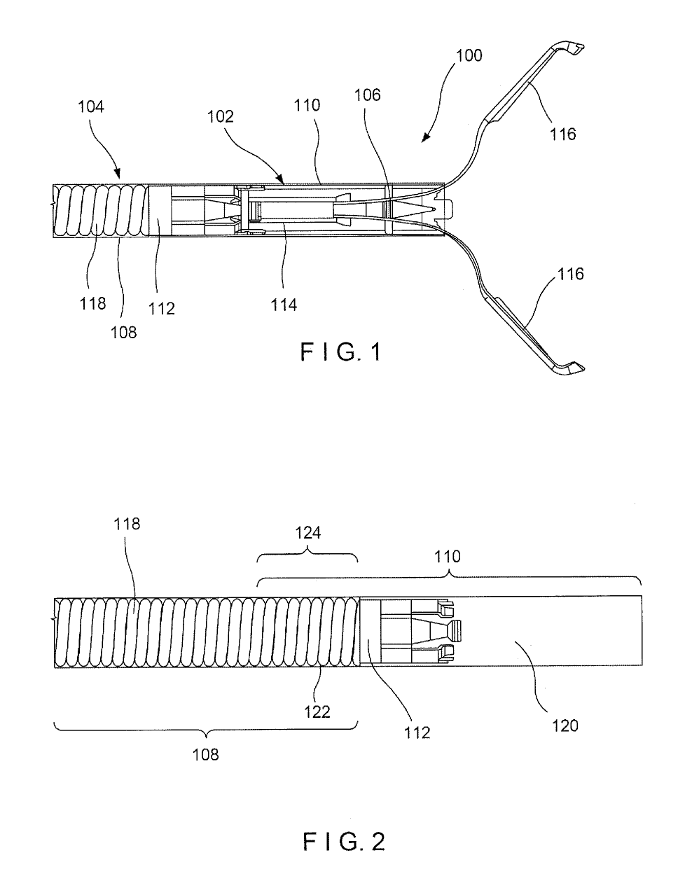

As shown in FIGS. 1-2, a clipping device 104 incorporating a system 100 according to an exemplary embodiment of the present invention comprises a mini sheath 102 covering a distal portion 106 thereof. For example, the clipping device 104 may be constructed, with the exception of the mini sheath 102, substantially as described in U.S. patent application Ser. No. 12/485,542, entitled “Hemostatic Clipping Devices and Methods” to Menn, et al., filed Jun. 16, 2009 and U.S. patent application Ser. No. 12/107,559 entitled “Single Stage Hemostasis Clipping Device” to Cohen et al., filed Apr. 22, 2008, the entire disclosures of which are hereby incorporated herein in their entireties. As would be understood by those skilled in the art, the distal portion 106 of such a clip including the clipping arms and a capsule holding the clipping arms has the most potential for damaging the scope if left uncovered. Specifically, the distal portion 106 of the clipping device 104 may include a bushing 112 releasably coupled to a capsule 114, which substantially houses arms 116 such that they are movable between an open configuration to receive tissue therebetween and a closed configuration to grip tissue. The bushing 112 may be connected to a handle (not shown) via a coil 118. In the open configuration, as shown in FIG. 1, the arms 116 extend distally beyond a distal end of the capsule 114. In the closed configuration, proximal portions of the arms 116 are drawn into and restrained by the capsule 114 with distal ends of the arms 116 projecting distally therefrom. Once the arms 116 have been drawn together gripping the target tissue, the capsule 114 is released from the bushing 112 leaving the capsule 114 over the arms and the arms locked closed over the gripped tissue. As shown in FIG. 2, in a deployed configuration, the capsule 114 is released from the bushing 112 which remains coupled to the coil 118. It will be understood by those of skill in the art, however, that the clipping device 104 may be any endoscopic device and that the distal portion 106 of the clipping device 104 may represent any portion of the endoscopic device having the potential to damage the channel of the endoscope through which it is passed.

As shown in FIGS. 1 and 2, the clipping device 104 includes a mini sheath 102 having a heat shrink member 108 and an extrusion 110. The extrusion 110 is formed, for example, as a single piece extending over a portion of the clipping device 104 having the most potential to damage the working channel of the scope through, for example, abrasion. The extrusion 110 may be an element extruded over the distal portion 106 with an outer surface thereof in a desired shape. For example, an outer surface of the extrusion 110 may be substantially cylindrical. However, it will be understood by those of skill in the art that the extrusion 110 may be formed in any of a variety of shapes and sizes corresponding to the working channel of an endoscope through which it is to be passed. The extrusion 110 may be any substantially tubular member formed in any of a variety of known manners. As would be understood by those skilled in the art, the heat shrink member 108 may be a substantially tubular element formed of a material which shrinks when heated so that the heat shrink member 108 molds around and grips a surface over which it is positioned when heated. In this case, the heat shrink member may be formed as a tube which, prior to heating, has an inner diameter slightly greater than an outer diameter of the surface over which it is to be applied. This allows the heat shrink member 108 to be slid over the coil 118 to a desired position for heating.

In a first exemplary embodiment, as shown in FIG. 2, the extrusion 110 is positioned over the distal portion 106 extending over the capsule 114, the bushing 112 and a distal portion of the coil 118. It will be understood by those of skill in the art that an inner lumen 120 of the extrusion 110 must be sized and shaped to allow the capsule 114 to be deployed therefrom—i.e., so that, when released from the bushing 112, the capsule 114 is completely separated from the device 104 and remains in the body as the device 104 is withdrawn therefrom. The heat shrink member 108 is secured over the distal portion of the coil 118 with a distal portion 122 of the heat shrink member 108 overlapping a proximal portion 124 of the extrusion 110. The mini sheath 102 may then be placed in a split-die bonder such that the heat shrink member 108 shrinks over the coil 118, providing a tight seal over the proximal portion 124 of the extrusion 110. The split dies bonder may also cause the extrusion 110 to reflow into the coil 118 enhancing a bond therebetween. For example, the extrusion 110 may reflow over approximately six to eight of the distal-most turns of the coil 118 while the heat shrink member 108 keeps the extrusion 110 in place. The heat shrink member 108 also provide an increased column strength over the distal portion 106, which may be preferred by users. In an alternative embodiment, the heat shrink member 108 may be a coating applied or melted over the distal end 106 of the clipping device 104. In another alternative embodiment the heat shrink member 108 may be friction fit with the tubing of the extrusion 110.

The heat shrink member 108 may be formed of any known biocompatible heat shrink material. In a preferred embodiment, the heat shrink member 108 may be formed of a thermoplastic material such as, for example, polyethylene terephthalate (PET). Alternatively, the heat shrink member 108 may be any tubing element formed of for example, a low density polyethylene (LDPE), a medium density polyethylene (MDPE), a high density polyethylene (HDPE) or any combination thereof. The extrusion 110 may also be formed of any suitable biocompatible extrudable material such as, for example, a thermoplastic. In a preferred embodiment, the extrusion 110 may be formed of a combination of an LDPE and HDPE blend. In an alternate embodiment, the extrusion 110 may be formed either LDPE or HDPE. The heat shrink 108 and/or the extrusion 110 of the mini sheath 102 may be formed of a radiopaque material and/or include a radiopaque pattern along a portion of a length thereof to facilitate observation of the distal end, as would be understood by those skilled in the art.

It will be understood by those of skill in the art that portions of the mini sheath 102 may be blunt, rounded, tapered, soft, etc. to facilitate insertion of the clipping device 104 through the endosocpe. The mini sheath 102 allows the flexibility of a proximal portion of the device 104 to be maintained while protecting a distal end of the endoscope. The system 100 may be easily manufactured due to the usage of less material for the mini sheath 102 and may therefore also result in lower costs. It will also be understood by those of skill in the art that the extrusion 110 may be any length, covering any portion of the hemostatic clipping device 104 considered to be abrasive while balancing the reduction in abrasion against the reduced flexibility of the section covered thereby.

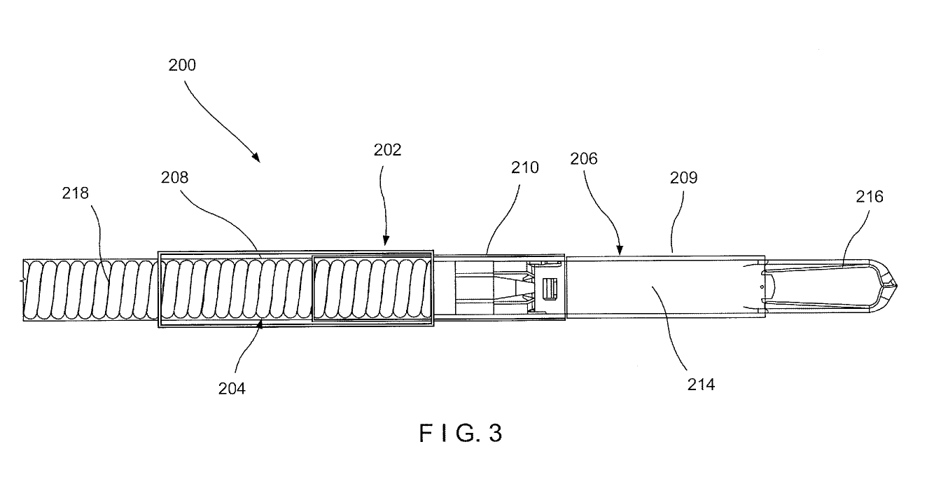

As shown in FIG. 3, a system 200 according to a second exemplary embodiment of the present invention may be substantially similar to the system 100, with a mini sheath 202 covering a distal portion 206 of a clipping device 204. Similarly to the mini sheath 102, the mini sheath 202 comprises a first heat shrink member 208 that holds an extrusion 210 over at least a portion of coils 218. The system 200, however, further comprises a second heat shrink member 209 that is completely shrunk over a capsule 214 of the clipping device 204 such that the second heat shrink member 209 will remain over the capsule 214 even after the clipping device 104 has been deployed. The first heat shrink member 208 and the extrusion 210 may be positioned over the distal portion 206, similarly to the heat shrink member 108 and the extrusion 210, described above in regard to the system 100. Thus, the first heat shrink 108 provides scope protection during insertion and removal while enhancing the column strength of the clipping device 104. The extrusion 210, however, may be shorter in length than the extrusion 110, covering only a proximal end of the bushing 212 and the capsule 214. A remaining portion of the capsule 214 may be covered by the second heat shrink member 209, which does not overlay any portion of a coil 218. The second heat shrink member 209 may be a thin material applied over leading edges of the capsule 214 and shrunk radially inward to secure the second heat shrink member 209 to the capsule 214. The second heat shrink member 209 remains attached to the capsule 214 during deployment and thus remains in the body along with the capsule 214 as long as the capsule 214 is attached to tissue within the body.

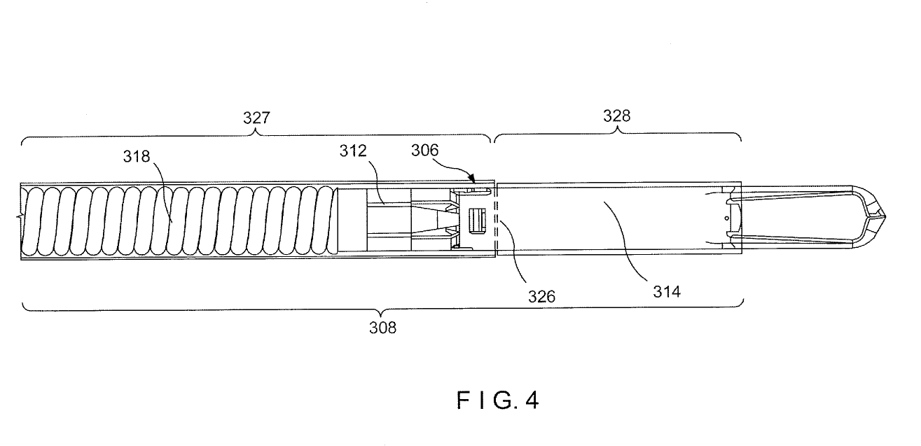

As shown in FIG. 4, a system 300 according to a third exemplary embodiment of the present invention is substantially similar to the systems 100, 200, as described above, but includes a mini sheath 302 including a heat shrink member 308 with no extrusion. The heat shrink member 308 is applied directly over a distal portion 306 of a clipping device 304. It will be understood by those of skill in the art that the heat shrink 308 may be applied to varying portions and lengths of the distal portion 306 covering, for example, the same areas covered by the mini sheaths of the previous embodiments. In a preferred embodiment, the heat shrink member 308 covers at least a portion of both a capsule 314 and a bushing 312 to provide enhanced column strength while protecting against scope abrasion. In an exemplary embodiment, the heat shrink 318 may be applied over a distal portion of the coils 318, the bushing 312 and leading edges of the capsule 314. A weakened section (e.g., perforations 326) is formed around the heat shrink member 308 at a location selected to facilitate separation of the capsule 314 from the bushing 312 after the clip has been deployed. For example, the weakened section may be formed around at least a portion of a perimeter of the heat shrink member 308 over the location at which the bushing 312 and the capsule 314 are releasably coupled to one another such that a distal portion 328 of the heat shrink member 308 covering the capsule 314 detaches from a proximal portion 327 thereof extending over the bushing 312 and, optionally a portion of the coil 318 when the clip is deployed. The cut 326 may be a perforation or a solid line at which the heat shrink member 308 is weakened or in any other suitable manner as would be understood by those skilled in the art. It will be understood by those of skill in the art that the location and shape of the cut 326 may vary according to a pattern and a location of detachment between the capsule 314 and the bushing 312 in any desired pattern.

It will be apparent to those skilled in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A medical device for insertion through a channel of an endoscope, comprising:
a sheath forming a smooth outer surface over a first abrasive portion of a length of the device, a second portion of the length of the device remaining uncovered by the sheath.

2. The device of claim 1, further comprising:
a first member extending over the first portion.

3. The device of claim 2, wherein the first member is heat shrink.

4. The device of claim 1, wherein the first abrasive portion is a distal portion of the device.

5. The device of claim 2, wherein the sheath includes an extrusion extending over at least a part of a distal portion of the length of the device, the first member overlapping at least a proximal part of the extrusion and extending radially outside the overlapped portion of the extrusion.

6. The device of claim 5, wherein the extrusion is substantially tubular and includes a lumen sized and shaped to accommodate the distal portion of the device.

7. The device of claim 2, wherein the first member includes a weakened portion at a location selected to facilitate use of a distal portion of the device from a portion of the device extending proximally therefrom.

8. The device of claim 2, wherein the first member comprises one of a polyethylene terephthalate, a low density polyethylene and a high density polyethylene.

9. The device of claim 2, further comprising:
a second member extending over a third portion of the length of the device that is releasable from the device.

10. The device of claim 9, wherein the second member is a heat shrink.

11. A hemostatic clipping device for insertion into a body via a channel of an endoscope, comprising:
a flexible insertion section including a hemostatic clip releasably coupled to a distal end of the insertion section, the sheath covering a distal portion of the insertion section and at least a portion of the clip.

12. The device of claim 11, wherein the sheath including a first member.

13. The device of claim 12, wherein the sheath further includes a tubular member around the distal end of the insertion section and at least a distal portion of the first member extending radially outside the tubular member.

14. The device of claim 12, wherein the first member is a heat shrink.

15. The device of claim 13, wherein the tubular member is formed of an extruded material.

16. The device of claim 12, wherein the first member includes a weakened portion extending therearound at a location selected to facilitate separation of the clip from the insertion section.

17. The device of claim 13, wherein the tubular member covers the distal end of the insertion section and a portion of the clip, the distal end of the insertion section including a bushing coupled to a distal end of a flexible coil, the first member extending over the distal end of the coil and a proximal portion of the tubular member, the tubular member including a lumen sized and shaped to slidably accommodate the clip such that the clip is releasable from within the tubular member when separated from the distal end of the insertion section.

18. The device of claim 12, wherein the first member includes one of a polyethylene terephthalate, a low density polyethylene and a high density polyethylene.

19. The device of claim 12, wherein the sheath includes a second member covering the hemostatic clip releasably coupled to the distal end of the insertion section.

20. The device of claim 19, wherein the tubular member is a heat shrink.

21. A sheath for a medical device extending from a distal end of the medical device along a portion of a length thereof to cover only a distal-most portion of the medical device.
